(19) United States
(12) Patent Application Publication
(10) Pub. No.: US 2013/0156644 A1
(43) Pub. Date: Jun. 20, 2013

(54) INTEGRATED MICROFLUIDIC CARTRIDGE

(71) Applicants: Woochang LEE, Anyang-si (KR); Youn Suk CHOI, Yongsin-si (KR); Yeol ho LEE, Seoul (KR); Young Ki HAHN, Seoul (KR); Soo Suk LEE, Suwon-si (KR)

(72) Inventors: Woochang LEE, Anyang-si (KR); Youn Suk CHOI, Yongsin-si (KR); Yeol ho LEE, Seoul (KR); Young Ki HAHN, Seoul (KR); Soo Suk LEE, Suwon-si (KR)

(73) Assignee: SAMSUNG ELECTRONICS CO., LTD., Suwon-si (KR)

(21) Appl. No.: 13/607,260

(22) Filed: Sep. 7, 2012

(30) Foreign Application Priority Data
Dec. 14, 2011 (KR) 10-2011-0134894

(51) Int. Cl. G01N 33/53; G01N 19/00; B01L 9/00; G01N 29/032; B82Y 15/00

(52) U.S. Cl. 422/69; 422/560; 977/810

(57) ABSTRACT

Embodiments of the disclosure describe a structure for mounting a biosensor in a microfluidic cartridge including a biosensor holder, a gasket seal, a strut, and a leaf spring using a method for maintaining constant pressure and a microfluidic cartridge including the same. The structure allows pressure to be evenly distributed to the biosensor in order to secure reproducibility and reliability of the detection signal, and enables integrated configuration of the cartridge by using a minimum amount of space.

### CROSS-REFERENCE TO RELATED APPLICATION

This application claims the benefit of Korean Patent Application No. 10-2011-0134894, filed on Dec. 14, 2011, and all the benefits accruing therefrom under 35 U.S.C. §119 which is hereby incorporated by reference as if fully set forth herein.

### BACKGROUND

1. Field

Provided are a structure for mounting a biosensor in a microfluidic cartridge, and a microfluidic cartridge including the same.

2. Description of the Related Art

Conventional biosensors perform quantitative or qualitative analysis and diagnosis on biological substances such as proteins, deoxyribonucleic acid (“DNA”), viruses, bacteria, cells, tissues, and so on by inducing a change in an electrical or optical signal using specific binding, reacting, etc. between the biological substances and a sensor surface. Detection of a biological substance requires a complex process for processing, reacting, and analyzing a reagent. Although the process depends upon the method of analysis and the type of material, a biosensor generally detects a biological substance through a complex combination of processes such as filtering, metering, mixing, transporting, reacting, and washing. In the case of conventional art, detection of a biological substance has been manually performed in respective laboratories using a variety of equipment. Fluid processing technology for automating and standardizing a test process has recently been developed along with biosensor technology. Technology for performing a process, which is currently performed manually in a clinical laboratory, in an automated single platform has been actively developed.

### SUMMARY

Provided is a structure for mounting a biosensor in a centrifugal force-based microfluidic cartridge for blood test, and a microfluidic cartridge including the same.

According to an aspect, disclosed is a structure for mounting a biosensor in a microfluidic cartridge comprising: a holder for the biosensor; a gasket seal for preventing leakage of fluid, which is contacted with the biosensor; a strut for distributably applying pressure to an entire area contacted with the biosensor; and a leaf spring for generating pressure in a direction perpendicular to the strut, which is deformed against a cartridge wall. The structure allows integrated configuration of the cartridge by using a minimum space.

According to another aspect, disclosed is a microfluidic cartridge comprising: a plasma separating part for separating plasma from blood; a fluid storing part for storing fluid; a fluid injecting part for injecting the fluid into the fluid storing part; and a biosensor mounted by means of the structure. The microfluidic cartridge employs a method for maintaining constant pressure so as to secure reproducibility and reliability of a detection signal.

### BRIEF DESCRIPTION OF THE DRAWINGS

The above and other aspects, advantages and features of this invention will become more apparent by describing in further detail exemplary embodiments thereof with reference to the accompanying drawings, in which:

FIG. 1 is an isometric view of a cartridge including a gasket seal-strut-leaf spring structure;

FIG. 2 is a cross-sectional view of the cartridge including the gasket seal-strut-leaf spring structure;

FIG. 3 is a top view of the microfluidic cartridge;

FIG. 4 is an isometric view of the microfluidic cartridge; and

FIG. 5 is a graph showing detection results using the microfluidic cartridge.

### DETAILED DESCRIPTION

The invention now will be described more fully hereinafter with reference to the accompanying drawings, in which a non-limiting embodiment is shown. This invention may, however, be embodied in many different forms, and should not be construed as limited to the exemplary embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated regions, integers, steps, operations, elements, and/or components, but do not preclude the presence or addition of one or more other regions, integers, steps, operations, elements, components, and/or groups thereof

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the disclosure, and will not be interpreted in an idealized or overly formal sense unless expressly so defined herein.

One or more embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear portions. Moreover, sharp angles that are illustrated may be rounded. Thus, the regions illustrated in the figures are schematic in nature and their shapes are not intended to illustrate the precise shape of a region and are not intended to limit the scope of the claims.

Example embodiments describe a structure for mounting a surface acoustic wave (“SAW”) biosensor in a centrifugal force based microfluidic cartridge for blood testing.

In a biosensor, such as a SAW sensor using a piezoelectric element, it is important to form micro-channels in a patterned piezoelectric element and mount the piezoelectric element. The piezoelectric element is sensitive to the amount of material present on its surface, external pressure applied to its surface, viscosity of fluid, and the like. Thus, when a tool such as a gasket is used to form the channels in the piezoelectric element, external pressure is applied to the piezoelectric element causing the sensor sensitivity to be changed. When the applied pressure of each sensor is changed, it is very difficult to get a constant response signal even when the same mass is introduced into the sensor surface.

When a mass sensor, such as a SAW sensor, is mounted to a microfluidic cartridge, a structure for mounting a sensor is employed to maintain constant pressure so as to secure reproducibility and reliability of a detected signal. For example, constant pressure can be maintained by diminishing a local deviation of pressure applied to a sensor by means of the structure for mounting a sensor described in various embodiments of this disclosure. Further, in consideration of mass production, blood collection of a patient, and non-specific adsorption reaction by cartridge materials, it is necessary to integrate cartridge components, including a fluid storing part and a valve, in order to minimize a size of the microfluidic cartridge. Space for mounting the biosensor in the microfluidic cartridge may be restricted by areas occupied by the cartridge components. A design for the cartridge structure capable of mounting the sensor in the restricted space is very important in reducing the size of the microfluidic cartridge.

Typically, as a method of mounting the biosensor in the cartridge, a screw method, a hook method, a bonding method using an adhesive or the like, a laminating method, an ultrasonic welding method, and the like have been used. Among these methods, the screw method may have different fastening pressures depending on positions because fastening forces are distributed by a fastening order of screws. The hook method may cause a difference in fastening force due to the deviation of an injected molding. The bonding method may cause a difference in fastening force caused by a variation when the adhesive is cured and by a remaining adhesive. The laminating method may cause delamination of a laminated interface by a pressure applied by high-speed rotation when fastened as well as contamination of a chip surface caused by an adhesive exposed by the introduction of fluid. The ultrasonic welding method may cause damage to a chip by excessive load when welded and deformation caused by the deviation of an injected molding when a welding instrument and a part are placed. Thus, a method of mounting the biosensor in a minimum space while applying constant pressure is required so that the biosensor is not interfered with by the other elements of the cartridge.

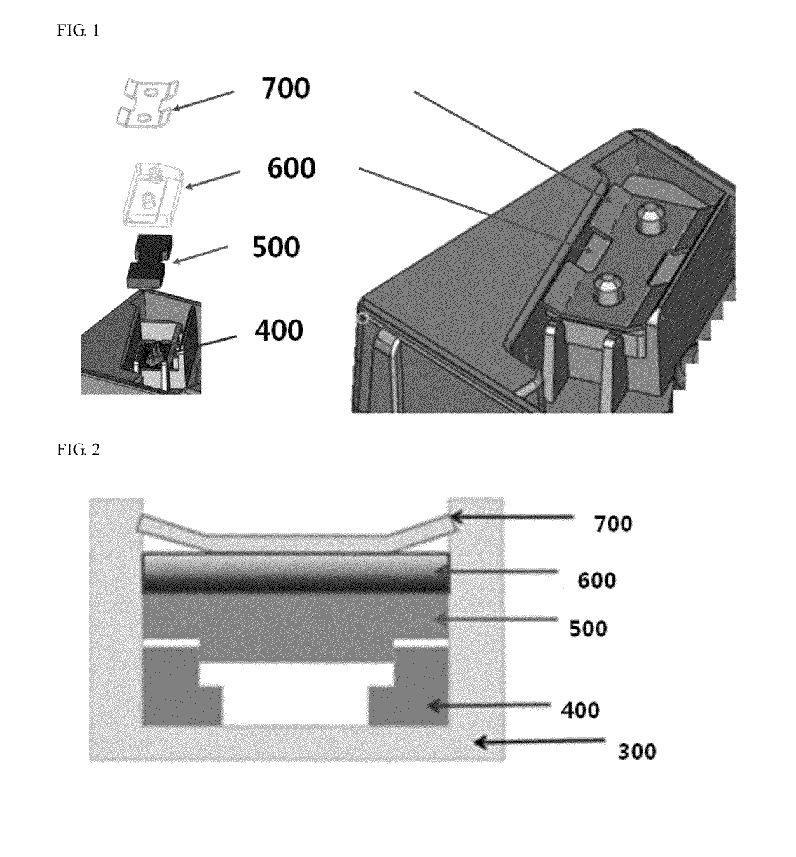

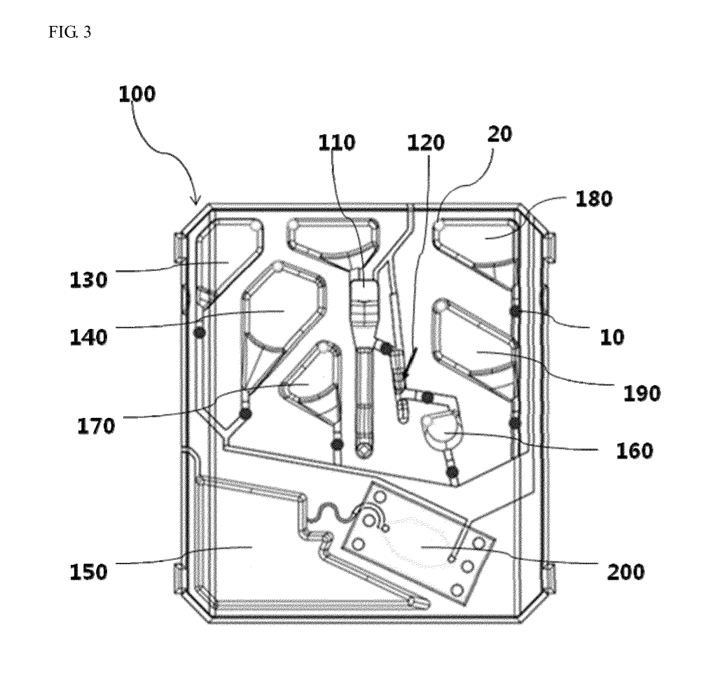

According to one embodiment, a structure is disclosed for mounting a biosensor in a microfluidic cartridge. The structure includes a gasket seal, a strut, and a leaf spring and employs a method for maintaining constant pressure, wherein the method may diminish local deviation of pressure applied externally to a surface of the biosensor. An isometric view and a cross-sectional view of the cartridge including the gasket seal 400, strut 600, and leaf spring 700 are shown in FIGS. 1 and 2, respectively.

As used herein, the term “cartridge” refers to an assembly of a chamber or a fluid path which is connected together as a single object that can be transferred or moved as one fitting. In the cartridge, at least some of parts such as a chamber may be firmly connected, whereas others such as a channel or a pipe connected to the chamber may be flexibly connected.

As used herein, the term “microfluidic cartridge” refers to a system or a device which includes at least one channel having microscopic dimensions and is used to treat, process, discharge, and analyze fluid. The term “channel” refers to a path which is formed in or through a medium enabling movement of fluid such as liquid or gas. The term “micro-channel” refers to a channel which is formed in a microfluidic system or device, and may have a cross section of about 1 mm², about 500 μm², about 100 μm², or about 50 μm². The micro-channel may take many forms. For example, the micro-channel may include a linear or non-linear array, and a U-shaped array. In an embodiment, the microfluidic cartridge 100 may be provided with an intagliated holder 300 in which the biosensor is placed. In an embodiment, the holder is formed by or located in a depression in the microfluidic cartridge 100.

As used herein, the term “method for maintaining constant pressure” refers to a method of diminishing the local deviation of pressure on a surface of the biosensor by evenly distributing external pressure across the entire area of the surface of the biosensor, so that the constant pressure may be maintained on the whole. Furthermore, an integrated structure of the cartridge can be achieved in a minimum space by using the method, and thus, a constant detection signal can be obtained conclusively.

As used herein, the term “gasket seal” refers to a mechanical sealing structure that generally fills a space between two or more coherent surfaces in order to prevent leakage from or to an assembly under pressure. The gasket seal 400 may be formed of, but is not limited to, an elastomer such as natural rubber, styrene-butadiene rubber, butadiene rubber, chloroprene rubber, nitrile rubber, nitrile butadiene rubber, butyl rubber, ethylene-propylene rubber, chlorosulfonated polyethylene rubber, acryl rubber, fluoro-rubber, silicon rubber, buna rubber, neoprene, or silicon. The elastomer may have Shore A hardness of about 50 to about 100, about 60 to about 90, or about 70 to about 80 as specified in Type A of ASTM D2240 05.

As used herein, the term “strut” refers to a plate structure that makes it possible to evenly distribute pressure to an entire area contacting the biosensor. A material of the strut 600 is not substantially limited as long as the strut can have a degree of flatness capable of coming into uniform contact with a rear face of the biosensor 500.

As used herein, the term “leaf spring” refers to a structure that is deformed against a cartridge wall and is able to apply pressure in a direction perpendicular to a surface of a strut, e.g., the top surface of the strut 600 illustrated in FIG. 2. In the embodiment illustrated by FIGS. 1 and 2, pressure is applied by the leaf spring 700 to the strut 600, which is then evenly distributed across a surface of the biosensor 500 by the strut 600. The leaf spring 700 may be made up of a flat central or intermediate portion providing a contact surface that contacts a surface of the strut 600, and one or more terminal portions (ends) bent at a predetermined inclination. In other words, the flat central or intermediate portion may be flanked, in whole or in part, by two or more terminal portions bent at a predetermined inclinantion relative to the plane of the central or intermediate portion. The terminal portion may have an angle equal to or less than about 45°, about 40°, or about 35° with respect to an extension line of the flat surface (plane) of the intermediate portion.

In some embodiments, the structure for mounting a biosensor in a microfluidic cartridge enables the mounting pressure to be maintained at a constant level by controlling an external pressure applied to the entire area of the biosensor. Further, the mounting structure minimizes interference with cartridge components in a restricted space, so that it is possible to inhibit geometrical expansion of the cartridge and to construct the cartridge in an integrated type.

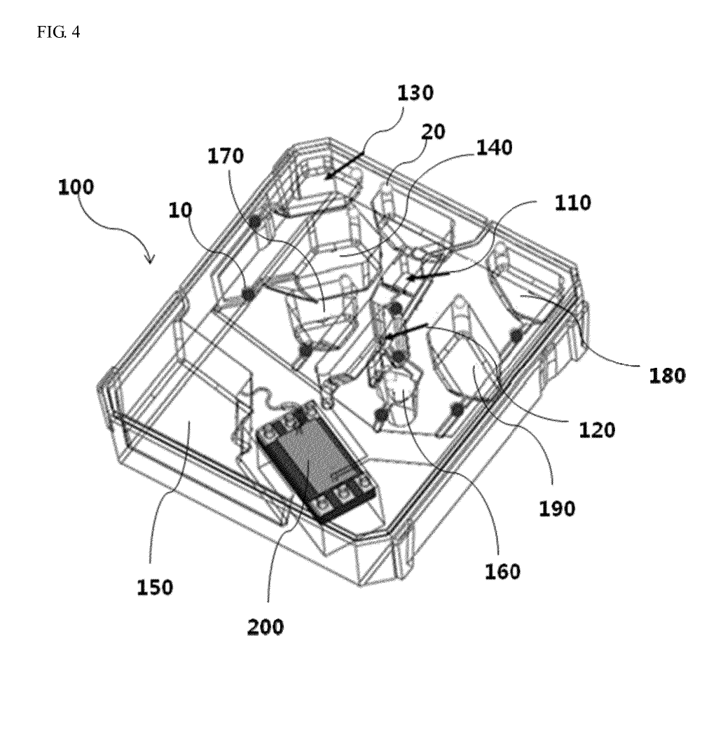

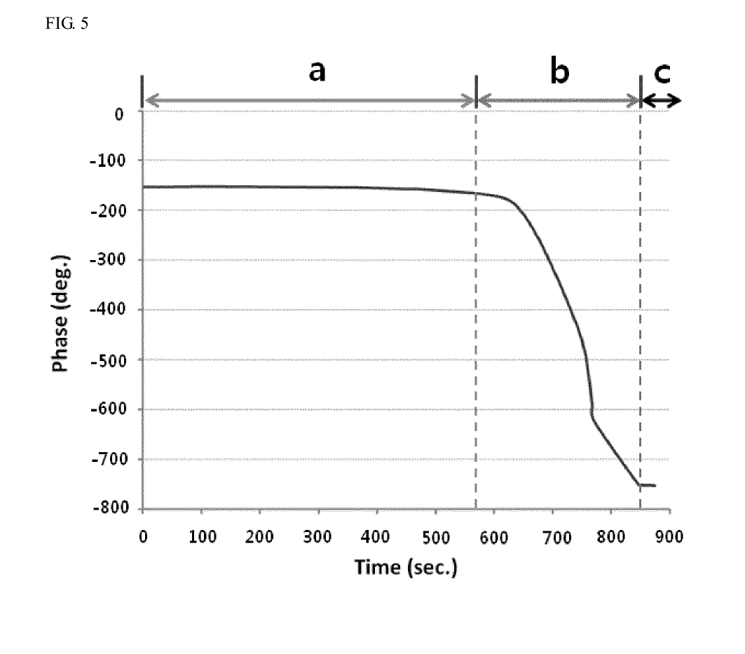

According to another embodiment, disclosed is a microfluidic cartridge including the mounting structure. The microfluidic cartridge may include a plasma separating part, a fluid injecting part, a fluid storing part, a fluidic valve, a biosensor, and a system for mounting the biosensor. A top view and an isometric view of the microfluidic cartridge are shown in FIGS. 3 and 4, respectively.

As used herein, the term “plasma separating part” refers to an area that is able to separate plasma from blood. The plasma separating part 110 may be able to separate the plasma by using a density difference between a corpuscle and the plasma and by using a centrifugal force caused by high-speed rotation of the cartridge 100. The plasma separated by the plasma separating part 110 may be stored in the plasma storing part.

As used herein, the term “fluid injecting part” refers to a portion for injecting fluid into a microfluidic channel (e.g., an injection port). The fluid refers to a material that is amorphous and has a flowing property. The fluid may include liquid and/or gas. For example, the fluid may include, but is not limited to, proteins, deoxyribonucleic acid (“DNA”), ribonucleic acid (“RNA”), peptides, carbohydrates, bacteria, plant, molds, animal cells, or surfactants.

As used herein, the term “fluid storing part” refers to an area where the fluid is able to stay for a predetermined time (e.g., a vessel or chamber). In the embodiment, the fluid storing part may include a plasma storing part 120, reagent storing parts 160, 180 and 190, and washer storing parts 130, 140 and 170. The plasma storing part 120 refers to an area for storing the plasma separated by the plasma separating part. The plasma storing part 120 may be connected with the plasma separating part 110 at an upper end thereof, and with the reagent storing parts at a lower end thereof

Each of the reagent storing parts 160, 180 and 190 refers to an area that is able to store a reagent for analysis or to mix the reagent with the plasma introduced from the plasma storing part. The first reagent storing part 160 may be connected with the plasma storing part 120 at an upper end thereof, and with a biosensor 200 at a lower end thereof When proteins in blood are detected using the microfluidic cartridge 100, the first reagent storing part 160 may store an adsorbent for adsorbing the proteins in blood. For example, the adsorbent may include, but is not limited to, gold nano-particles with a nano size. The second reagent storing parts 180 and 190 refer to areas for storing an additional reagent in order to improve detection sensitivity of biological substances. When the gold nano-particles for adsorbing proteins are stored in the first reagent storing part 160 of the microfluidic cartridge 100, the second reagent storing parts 180 and 190 may store a reagent capable of improving the detection sensitivity by increasing the mass of the gold nano-particles. For example, the second reagent storing parts 180 and 190 may be stored with HAuCl₄.3H₂O or NH₂O.HCL.

Each of the washer storing parts 130, 140 and 170 refers to an area for storing fluid for washing the biosensor 200.

As used herein, the term “fluidic valve” refers to a valve that is installed on a channel to control a flow of fluid. The fluidic valve 10 is a closed valve that blocks the flow of fluid and may be opened by external energy. The external energy may be, for instance, electromagnetic waves. An energy source may be a laser light source for irradiating laser beams, an emission device for irradiating visible or infrared light, or a xenon lamp. A source of the external energy may be selected according to a wavelength of the electromagnetic waves which may be absorbed by heating particles included in a material of the valve. The material of the valve may be a phase-change material whose phase varies with energy or a thermoplastic resin. The phase-change material may be, for instance, wax or gel. Also, the material of the valve may include micro-heating particles distributed in a phase-change material and used to absorb energy of the electromagnetic waves and generate heat. The micro-heating particles may include, but are not limited to, metal oxide particles such as Al₂O₃, TiO₂, Ta₂O₃, Fe₂O₃, Fe₃O₄, or HfO₂, polymer particles, quantum dots, or magnetic beads.

As used herein, the term “biosensor” refers to a device that is able to perform quantitative or qualitative analysis and diagnosis on biological substances by inducing a change in a signal, e.g., an electrical or an optical signal, using specific binding, reaction, etc. between the biological substances and a sensor surface. The biosensor 200 is connected with the fluid storing parts 130, 140, 160, 170, 180 and 190 at a front end thereof, and with a waste storing part 150 at a rear end thereof. The waste storing part 150 refers to an area for storing and discharging all waste after the detection of the biosensor 200.

The biosensor may be classified into a mass-based sensor, an optical sensor, an electrical sensor, and a magnetic force-based sensor. The mass-based sensor may include a quartz crystal microbalance (“QCM”), a cantilever sensor, and a SAW sensor. The optical sensor may include sensors using UV-visible spectrometry, colorimetry, and surface plasmon resonance (“SPR”). The electrical sensor may include an electrochemical sensor and a field effect transistor (“FET”) sensor. The magnetic force-based sensor may include a magnetic force microscope (“MFM”). In an example embodiment, a mass-based sensor is used. SAW refers to an acoustic wave propagated along the surface of a piezoelectric material, and may sense genes or proteins using a principle that a strong interaction with a medium abutting the piezoelectric material produces more influence on speed and amplitude of the acoustic wave.

The microfluidic cartridge may include, but is not limited to, an inorganic material such as glass or silicon as well as a polymer material such as silicon rubber, isobornyl acrylate, polyethylene terephthalate, poly dimethyl siloxane, poly methyl methacrylate, polycarbonate, polypropylene, polystyrene, polyvinyl chloride, polysiloxane, polyimide, or polyurethane.

The microfluidic cartridge may be manufactured by a laminating method, a bonding method based on an adhesive and surface modification, or an ultrasonic welding method. For example, the microfluidic cartridge 100 may be formed of polystyrene, and include the fluid injecting part 20 at an upper layer thereof and the fluid storing parts 120, 130, 140, 160, 170, 180, and 190 and the micro-channel at a lower layer thereof. The fluid injecting part 20, fluid storing parts 120, 130, 140, 160, 170, 180, and 190, and the micro-channel may be formed by a typical computer numerical control (“CNC”) machine. The upper and lower layers may be adhered by ultrasonic welding. The microfluidic device may have peripheral dimensions of 40.0×43.0×9.5 mm³. A rotary substrate for installing the microfluidic cartridge may be manufactured in the same method as described above.

A ferro-wax valve 10 may be installed on an upper end of the channel to control a flow of fluid. Ferro wax may be heated at a temperature equal to or higher than about 80° C., and then provided to the lower ends of the fluid storing parts 120, 130, 140, 160, 170, 180, and 190. When injected into the lower ends of the fluid storing parts 120, 130, 140, 160, 170, 180, and 190, the ferro wax may move into the channel by means of a capillary force, and be rapidly solidified due to the radiation of heat.

An example process of detecting biological substances using the microfluidic cartridge is described below.

Blood is injected into the plasma separating part 110, and then plasma separated from the blood is collected in the plasma storing part 120. Meanwhile, reagents have been stored in the first reagent storing part 160 and the second reagent storing parts 180 and 190.

For example, when proteins are intended to be detected from the blood, the first reagent storing part 160 stores detection antibody-gold nano-particles for coupling with the proteins in the plasma, whereas the second reagent storing parts 180 and 190 may store HAuCl₄.3H₂O and NH₂OH.HCl, respectively, for increasing the mass of the gold nano-particles to improve detection sensitivity. When the plasma is collected in the plasma storing part 120, the fluidic valve 10, the plasma storing part 120, and the first reagent storing part 160 are rotated. Thereby, the fluidic valve 10 is opened, and the plasma in the plasma storing part 120 is injected into the first reagent storing part 160 in which the detection antibody-gold nano-particles are stored.

The plasma supplied to the first reagent storing part 160 and the detection antibody-gold nano-particles are mixed while moving to the micro-channel. The proteins in the plasma are adsorbed by the detection antibody-gold nano-particles. The reagent mixture discharged from the first reagent storing part 160 is injected into the biosensor 200. At this time, a surface of the biosensor 200 is stabilized by a buffer stored in the washer storing part 130.

The biosensor 200, into which the reagent mixture is injected, is washed with the buffer stored in the washer storing part 130, thereby removing any abnormally remaining detection antibody-gold nano-particles other than the detection antibody-gold nano-particles normally coupled to the sensor surface. This washing process may achieve an increase in detection precision. Both the reagent mixture, which is not coupled to the sensor surface, and the buffer are stored in the waste storing part 150, or discharged. Then, when the valves of the second reagent storing parts 180 and 190 are open, HAuCl₄.3H₂O and NH₂OH.HCl stored in the respective second reagent storing parts 180 and 190 are mixed in one of the second reagent storing parts 180 and 190, and then the mixture is injected into the biosensor 200 by opening the valves in turn. The reagent mixture coupled to the surface of the biosensor 200 undergoes a redox reaction with the gold nano-particles, thereby increasing the mass of the gold nano-particles. Thus, a detection signal of the biosensor 200 is amplified. Finally, the biosensor 200 is washed with the buffer stored in the washer storing part 170, and performs sensing. Then, it is possible to measure a change in phase in the biosensor.

The microfluidic cartridge structure including the gasket seal 400, the strut 600, and the leaf spring 700 can provide reproducibility and reliability of the detection signal by using the method for maintaining constant pressure. Further, the size of the microfluidic cartridge structure can be minimized by integrating the elements of the cartridge.

### Embodiment: Analysis results of cTnI Using the Biosensor

cTnI (Cardiac troponin I) of a concentration of 25 ng/mL is analyzed by using the biosensor mounted in the microfluidic cartridge with the method for maintaining constant pressure. The analysis results are shown in FIG. 5. In FIG. 5, “a” represents a sensor surface reaction step, “b” represents a signal amplification reaction step, and “c” represents a final washing and detection step. As can be seen from FIG. 5, the sensor surface reaction step a shows a weak phase change, whereas the additional reaction process of increasing the mass, i.e. the signal amplification reaction step b shows a strong phase change.

Accordingly, the microfluidic cartridge according to the embodiment can provide a uniform distribution of the external pressure applied to the entire area of the biosensor so that a constant pressure may be maintained on the whole, and regulate constant load, so that the microfluidic cartridge can contribute to improving the reproducibility and reliability of the biosensor.

While the invention has been particularly shown and described with reference to embodiments thereof, it will be understood by those of ordinary skill in the art that various changes in form and details may be made therein without departing from the spirit or scope of the invention as defined by the following claims.

What is claimed is:

1. A system for mounting a biosensor in a microfluidic cartridge, the structure comprising:
a holder for holding a biosensor;
a gasket seal for preventing leakage of fluid, the gasket seal in contact with the biosensor when present in the holder;
a strut for evenly distributing pressure across a surface of the biosensor when present in the holder; and
a leaf spring for applying pressure to the strut in a direction perpendicular to a surface of the strut, wherein the leaf spring is deformed against a wall of the holder.

2. The system according to claim 1, wherein the holder is an intagliated holder in a microfluidic cartridge.

3. The system according to claim 1, wherein the microfluidic cartridge is manufactured by a laminating method, a bonding method based on an adhesive and surface modification, or an ultrasonic welding method.

4. The system according to claim 1, wherein the microfluidic cartridge comprises at least one material selected from the group consisting of silicon rubber, isobornyl acrylate, polyethylene terephthalate, poly dimethyl siloxane, poly methyl methacrylate, polycarbonate, polypropylene, polystyrene, polyvinyl chloride, polysiloxane, polyimide, and polyurethane.

5. The system according to claim 1, wherein the gasket seal comprises at least one material from the group consisting of natural rubber, styrene-butadiene rubber, butadiene rubber, chloroprene rubber, nitrile rubber, nitrile butadiene rubber, butyl rubber, ethylene-propylene rubber, chlorosulfonated polyethylene rubber, acryl rubber, fluoro-rubber, silicon rubber, buna rubber, neoprene, and silicon.

6. The system according to claim 5, wherein the gasket seal has Shore A hardness of about 50 to about 100.

7. The system according to claim 1, wherein the leaf spring includes:
a flat intermediate portion in contact with the surface with the strut: and
one or more terminal portions bent at a predetermined inclination relative to the flat intermediate portion.

8. The system according to claim 7, wherein the terminal portion of the leaf spring has an angle equal to or less than about 45° with respect to an extension line of the flat intermediate portion.

9. A microfluidic cartridge comprising:
a plasma separating part for separating plasma from blood;
a fluid storing part for storing fluid;
a fluid injecting part for injecting the fluid into the fluid storing part;
a biosensor; and
a system for mounting the biosensor according to claim 1.

10. The microfluidic cartridge according to claim 9, further comprising a fluidic valve that is installed in or on a channel and controls a flow of the fluid.

11. The microfluidic cartridge according to claim 9, further comprising a waste storing part for storing and discharging waste passing through the biosensor.

12. The microfluidic cartridge according to claim 9, wherein the fluid storing part includes a plasma storing part, a reagent storing part, and a washer storing part.

13. The microfluidic cartridge according to claim 12, wherein the reagent storing part comprises at least two storing parts, the reagent storing parts comprising at least one of an adsorbent for adsorbing the fluid, and a reagent for improving the detection sensitivity by the increased mass of the adsorbent.

14. The microfluidic cartridge according to claim 9, wherein the microfluidic cartridge is configured to be driven by a centrifugal force.

15. The microfluidic cartridge according to claim 9, wherein the fluid includes at least one selected from the group consisting of proteins, DNA, RNA, peptides, carbohydrates, bacteria, plant, molds, animal cells, and surfactants.

16. The microfluidic cartridge according to claim 9, wherein the biosensor is a mass-based sensor.

17. The microfluidic cartridge according to claim 16, wherein the biosensor is a quartz crystal microbalance, a cantilever sensor, or a surface acoustic wave sensor.

18. The microfluidic cartridge according to claim 9, wherein the microfluidic cartridge comprises at least one material selected from the group consisting of silicon rubber, isobornyl acrylate, polyethylene terephthalate, poly dimethyl siloxane, poly methyl methacrylate, polycarbonate, polypropylene, polystyrene, polyvinyl chloride, polysiloxane, polyimide, and polyurethane.

19. The microfluidic cartridge according to claim 9, wherein the microfluidic cartridge is manufactured by a laminating method, a bonding method based on an adhesive and surface modification, or an ultrasonic welding method.
